(19) United States
(12) Patent Application Publication
(10) Pub. No.: US 2024/0415785 A1
(43) Pub. Date: Dec. 19, 2024

(54) USE OF D-ARABITOL IN LIPID-LOWERING AND HEPATOPROTECTION

(71) Applicant: NANJING UNIVERSITY OF CHINESE MEDICINE, Nanjing (CN)

(72) Inventors: Jinjun SHAN, Nanjing (CN); Liuqing DI, Nanjing (CN); Zichen LUO, Nanjing (CN); Weichen XU, Nanjing (CN); Tong XIE, Nanjing (CN); Chen SHI, Nanjing (CN); Tianzi JIN, Nanjing (CN)

(73) Assignee: NANJING UNIVERSITY OF CHINESE MEDICINE, Nanjing (CN)

(21) Appl. No.: 18/815,905

(22) Filed: Aug. 27, 2024

Related U.S. Application Data
(63) Continuation-in-part of application No. PCT/CN2023/077120, filed on Feb. 20, 2023.

(30) Foreign Application Priority Data
Mar. 1, 2022 (CN) 202210194670 .6

(51) Int. Cl. A61K 31/047; A61K 9/00; A61P 1/16; A61P 3/06

(52) U.S. Cl. CPC A61K 31/047; A61K 9/0019; A61K 9/0056; A61P 1/16; A61P 3/06

(57) ABSTRACT

A use of D-arabitol in lipid-lowering and hepatoprotection is provided. Blood or liver lipid abnormalities refer to abnormalities in a quantity and a quality of lipids in the blood or liver, and usually refer to the elevation of cholesterol and/or triglycerides (TGs). Blood and liver lipid abnormalities can lead to atherosclerosis, increase the morbidity and mortality rates of cardio-cerebrovascular diseases, and cause the liver damage. D-arabitol has an excellent lipid-lowering and hepatoprotective effect, and has a prospect of being developed into a drug or health care product or food for lipid-lowering and hepatoprotection.

### CROSS REFERENCE TO THE RELATED APPLICATIONS

This application is a continuation-in-part application of International Application No. PCT/CN2023/077120, filed on Feb. 20, 2023, which is based upon and claims priority to Chinese Patent Application No. 202210194670.6, filed on Mar. 1, 2022, the entire contents of which are incorporated herein by reference.

### TECHNICAL FIELD

The present disclosure relates to a novel use of a known compound, and specifically to a use of D-arabitol in lipid-lowering and hepatoprotection.

### BACKGROUND

Blood or liver lipid abnormalities refer to abnormalities in quantity and quality of lipids in the blood or liver, and usually refer to the elevation of cholesterol and/or triglycerides (TGs). Blood and liver lipid abnormalities can lead to atherosclerosis, increase the morbidity and mortality rates of cardio-cerebrovascular diseases, and cause the liver damage.

D-arabitol, as a functional five-carbon sugar alcohol, has been used in some industries. In the food industry, D-arabitol can be used not only as a high-grade sweetener, but also as a matrix for preparing a syrup to improve a quality of an alcoholic beverage. In the pharmaceutical field, D-arabitol can be used as an intermediate for drugs such as vidarabine, cytarabine, and α-glucosidase inhibitors, and can also serve as a transport medium to cross the blood-brain barrier. In the chemical industry, D-arabitol can be used as a dissolution promoter for granular solids or hydrophilic coatings, and can enhance the reliability of an aluminum capacitor at a high temperature and improve a viscosity of an electrolyte solution. In addition, D-arabitol can also be used as an activator for synthesizing a polymer foam material and a stabilizer for a developing material. In the biological field, D-arabitol can also promote the plant growth.

Currently, there are no reports on the use of D-arabitol in lipid-lowering and hepatoprotection.

### SUMMARY

An objective of the present disclosure is to provide a use of D-arabitol in lipid-lowering and hepatoprotection.

The objective of the present disclosure is allowed through the following technical solutions:

A use of D-arabitol in preparation of a drug or health care product or food for lipid-lowering and hepatoprotection is provided.

A drug or health care product or food for lipid-lowering and hepatoprotection is provided, including D-arabitol as an active ingredient and an adjuvant acceptable in the drug or health care product or food.

A lipid-lowering method is provided, including: orally administering or injecting a drug to or into a patient with a high blood or liver lipid level, where an active ingredient of the drug is D-arabitol.

A hepatoprotection method is provided, including: orally administering or injecting a drug to or into a patient with a liver damage caused by a high blood or liver lipid level, where an active ingredient of the drug is D-arabitol.

A lipid-lowering method is provided, including: allowing an individual with a high blood or liver lipid level to take a health care product or a food, where the health care product or the food includes D-arabitol as an active ingredient.

A hepatoprotection method is provided, including: allowing an individual with a liver damage caused by a high blood or liver lipid level to take a health care product or a food, where the health care product or the food includes D-arabitol as an active ingredient.

### Beneficial Effects

The present disclosure has found that D-arabitol has an excellent lipid-lowering and hepatoprotective effect, and has a prospect of being developed into a drug or health care product or food for lipid-lowering and hepatoprotection.

### BRIEF DESCRIPTION OF THE DRAWINGS

FIG. 1 shows a change of a body weight ratio of mice in each group (n=8), where compared with a normal diet group, ***P<0.001; and compared with a high-fat diet group, ##P<0.01;

FIGS. 2A-2C show the comparison of blood lipid levels in mice among groups (n=6), where FIG. 2A shows total TG contents in serum; FIG. 2B shows low-density lipoprotein cholesterol (LDL-c) contents in serum; FIG. 2C shows high-density lipoprotein cholesterol (HDL-c) contents in serum; and compared with the high-fat diet group, *P<0.05, **P<0.01, and ***P<0.001;

FIG. 3 shows the comparison of total cholesterol (TC) levels in livers of mice among groups (n=6), where compared with the high-fat diet group, ***P<0.001;

FIG. 4 shows a hematoxylin and eosin (H&E) staining result of a mouse liver tissue in each group; and

FIG. 5 shows an alanine aminotransferase (ALT) level in mouse serum of each group (n=6), where compared with the high-fat diet group, ***P<0.001.

### DETAILED DESCRIPTION OF THE EMBODIMENTS

The substantive content of the present disclosure will be specifically described below in conjunction with examples, but the protection scope of the present disclosure is not limited thereto.

### Example 1

### I. Experimental Materials

### 1. Instruments

A JJ-12J dehydrator, a JB-P5 embedding machine, a JB-L5 freezing table (Wuhan Junjie Electronics Co., Ltd.), an RM2016 microtome, an LEICA 819 microtome blade (Shanghai Leica Instrument Co., Ltd.), a KD-P tissue water bath-slide dryer (Zhejiang Jinhua Kedee Instrumental Equipment Co. Ltd.), a GFL-230 oven (Tianjin Labotery Instrument Co., Ltd.), an Eclipse E100 upright optical microscope (Nikon, Japan), a Revco UXF ultra-low temperature freezer (Thermo Fisher Scientific, the United States), an M200 microplate reader (TECAN, the United States), a MinSpin high-speed centrifuge (Eppendorf, Germany), an MB100-4P thermostatic shaker (Hangzhou Allsheng Instruments Co., Ltd.), a CPA225D electronic balance (Sartorius, Germany), and an MM400 refrigerated mixer mill (Retsch, Germany).

### 2. Reagents and Materials

Absolute ethanol, xylene, a neutral gum, hydrochloric acid, and ammonia water: purchased from Sinopharm Chemical Reagent Co., Ltd. An eosin staining solution, a differentiation solution, a bluing solution, hematoxylin, and gelatin glycerin: purchased from Wuhan Servicebio Technology Co., Ltd. An HDL-c kit (20191209), an LDL-c kit (20191223), a TC kit (20191209), a total TG kit (20191209), and an ALT (alanine transaminase) kit (20191227): purchased from Nanjing Jiancheng Institute of Biological Engineering. D-arabitol (E34RF-MV): purchased from TCI (Shanghai) Development Co., Ltd. L-arabitol (A1921101): purchased from Shanghai Aladdin Biochemical Technology Co., Ltd. L-arabinose (LG30S37) and D-arabinose (LM50S07): purchased from Beijing J&K Scientific Ltd. Xylitol (C11954123): purchased from Shanghai Maclin Biochemical Technology Co., Ltd. A high-fat feed (D12492): purchased from Research Diets of the United States. A conventional feed (SWS9102): purchased from Jiangsu Xietong Pharmaceutical Bio-engineering Co., Ltd.

### II. Experimental Method

### 1. Drug Preparation

D-arabitol, L-arabitol, L-arabinose, D-arabinose, and D-xylitol each were prepared with sterile distilled water into a 50 g/L or 20 g/L solution.

### 2. Animals and Grouping

A total of 64 SPF-grade 8-week-old C57 BL/6 J male mice were adaptively raised for 3 d with a normal diet in a barrier facility having an independently-ventilated cage (IVC), tested for an initial body weight, and randomly divided into 8 groups. On day 4, the mice in the 8 groups were administered with a normal feed or a high-fat feed, and drinking water for mice in an administration group was replaced with drug-containing water. Each mouse had free access to food and drinking water, and a body weight was recorded weekly. The grouping, diets, and administration were as follows:
(1) normal diet group (Chow): normal feed+sterile distilled water;
(2) high-fat diet group (HFD): high-fat feed+sterile distilled water;
(3) 50 g/L L-arabitol group (HFD+50 g/L L-Arabitol): high-fat feed+50 g/L L-arabitol;
(4) 20 g/L D-arabitol group (HFD+20 g/L D-Arabitol): high-fat feed+20 g/L D-arabitol;
(5) 50 g/L D-arabitol group (HFD+50 g/L D-Arabitol): high-fat feed+50 g/L D-arabitol;
(6) 50 g/L L-arabinose group (HFD+50 g/L L-Arabinose): high-fat feed+50 g/L L-arabinose;
(7) 50 g/L D-arabinose group (HFD+50 g/L D-Arabinose): high-fat feed+50 g/L D-arabinose; and
(8) 50 g/L xylitol group (HFD+50 g/L Xylitol): high-fat feed+50 g/L xylitol.

### 3. Sample Collection

At the end of the experiment, each mouse was anesthetized by intraperitoneal injection of sodium pentobarbital with a concentration of 5 mg/mL, the injection volume was 0.1 mL/10 g, and the injection dose was 50 mg/kg, and then blood was collected from the orbit. The mouse was sacrificed through cervical dislocation, and a liver was collected, rinsed with normal saline, and dried with a filter paper. A liver lobe was taken and fixed in paraformaldehyde. The remaining liver was quickly frozen with liquid nitrogen, and stored at −80° C. The blood was allowed to stand for 30 min and then centrifuged at 3,500 rpm for 10 min to obtain a supernatant, and the supernatant was collected and stored at −80° C.

### 4. Histopathological Detection

A liver tissue that had been fixed with 4% paraformaldehyde was taken and subjected to gradient dehydration in the dehydrator as follows: in 75% ethanol for 4 h, in 85% ethanol for 2 h, in 90% ethanol for 2 h, in 95% ethanol for 1 h, in absolute ethanol I for 30 min, and in absolute ethanol II for 30 min. A tissue obtained after complete dehydration was permeabilized 1 time with a mixed solution of absolute ethanol and xylene mixed in equal volumes, and then permeabilized 2 times with pure xylene for about 5 min to 10 min each time. A tissue sample obtained after the permeabilization was soaked in pure paraffin 3 times for 1 h each time and then embedded with a melted paraffin on the embedding machine, and the melted paraffin was cooled and solidified on the freezing table at −20° C. A cooled paraffin block was fixed, trimmed, and sectioned with the microtome. A section was allowed to float on warm water at 40° C. in the water bath-slide dryer, spread, scooped up with a glass slide, baked in a 60° C. oven until water was completely evaporated and the paraffin was melted, taken out, and stored at room temperature for later use.

Before staining, a paraffin section was subjected to dewaxing and rehydration as follows: The paraffin section was treated with pure xylene 2 times for 20 min each time, then subjected to a gradient treatment with 100% ethanol, 90% ethanol, and 75% ethanol each for 5 min, and then washed with water. A section obtained after the dewaxing and rehydration was first soaked in the hematoxylin staining solution for 3 min to 5 min, washed with water, then differentiated with the differentiation solution, washed with water, then blued in the bluing solution, and rinsed with running water. After the hematoxylin staining was completed, a section was subjected to gradient dehydration with 85% ethanol and 95% ethanol each for 5 min, and then stained in the eosin staining solution for 5 min. After the staining was completed, a section was treated with absolute ethanol 3 times and xylene 2 times for 5 min each time, and finally mounted with the neutral gum.

The microscopic examination was conducted under a microscope. In the liver tissue, the nucleus was blue, the cytoplasm was red, and the fat droplet was a white vacuole.

### 5. Biochemical Indicator Detection

A serum sample was taken, thawed at 4° C., and tested for serum HDL-c, LDL-c, total TG, ALT, and aspartate aminotransferase (AST) according to instructions of kits.

A liver tissue sample was taken and thawed at 4° C. An appropriate amount of the liver tissue sample was weighed, absolute ethanol was added at an amount 9 times (v/m) of the amount of the liver tissue sample, and grinding was conducted in the refrigerated mixer mill for 5 min to obtain a homogenate. The homogenate was centrifuged at 2,500 rpm for 10 min, and tested for TC according to instructions of a kit.

### 6. Data Processing and Statistical Analysis

Mouse body weight ratio: a body weight at the nth week (g)×100%/an initial body weight (g). Data was subjected to statistical tests with Graphpad Prism v9.0. Two-way ANOVA and Tukey's multiple comparison tests were adopted for body weight data, and one-way ANOVA and Dunnett's multiple comparison tests were adopted for other data.

### III. Experimental Results

### 1. Body Weight

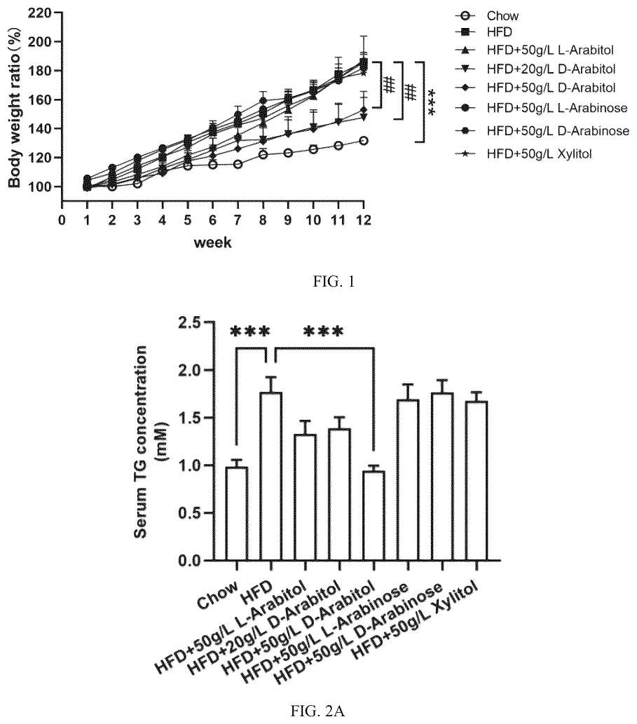

A trend of body weight changes of mice in each group was shown in FIG. 1. The growth of body weights of mice with the high-fat diet was significantly faster than the growth of body weights of mice with the normal diet, where a significant difference in a body weight ratio started from week 3 (P<0.05) and there was an extremely-significant difference (P<0.001) at week 12. Compared with mice of a model group: The administration of 50 g/L D-arabitol made a body weight ratio of high-lipid mice start to significantly decrease from week 4 (P<0.05), and P=0.0058 at week 12. The administration of 20 g/L D-arabitol made a body weight ratio of high-lipid mice start to significantly decrease from week 8 (P<0.05), and P=0.0060 at week 12. It indicates that D-arabitol can reduce the body weight gain of high-lipid mice in a concentration-dependent manner. However, the administration of 50 g/L L-arabitol, 50 g/L L-arabinose, 50 g/L D-arabinose, and 50 g/L xylitol did not have an effect of making a body weight ratio of mice significantly decrease.

### 2. Blood Lipid

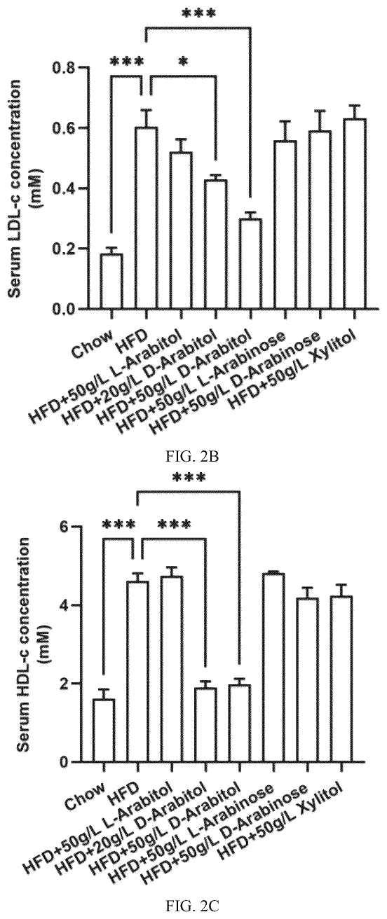

As shown in FIG. 2A: Compared with a normal diet group, the serum total TG in mice of the high-fat diet group increased significantly (P<0.001). The total TG in serum of mice with the high-fat diet that were administered with 50 g/L D-arabitol decreased significantly (P<0.001), and the total TG in serum of mice of other groups did not change significantly. As shown in FIGS. 2B-2C: LDL-c and HDL-c levels in mice of the high-fat diet group were significantly higher than LDL-c and HDL-c levels in mice of the normal diet group (P<0.001). The administration of 50 g/L or 20 g/L D-arabitol could significantly reduce the LDL-c and HDL-c concentrations in serum of high-lipid mice (P<0.05), and there was no significant change in other groups. It indicates that D-arabitol has a significant improvement effect for a blood lipid level in mice with the high-fat diet in a specified concentration-dependent manner.

### 3. Liver Lipid

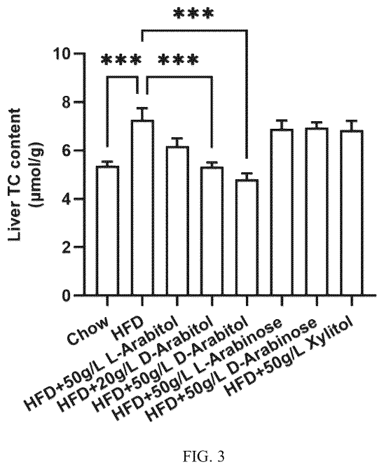

As shown in FIG. 3: A TC level in the liver of mice fed with the high-fat diet for 12 weeks significantly increased (P<0.001). The administration of 20 g/L or 50 g/L D-arabitol could significantly reduce the accumulation of TC in the liver of mice with the high-fat diet (P<0.01), and there was No Significant Change in Other Groups. It Indicates that D-Arabitol has a Significant improvement effect for the accumulation of liver lipids in mice with the high-fat diet in a specified concentration-dependent manner.

### 4. Liver Histopathology

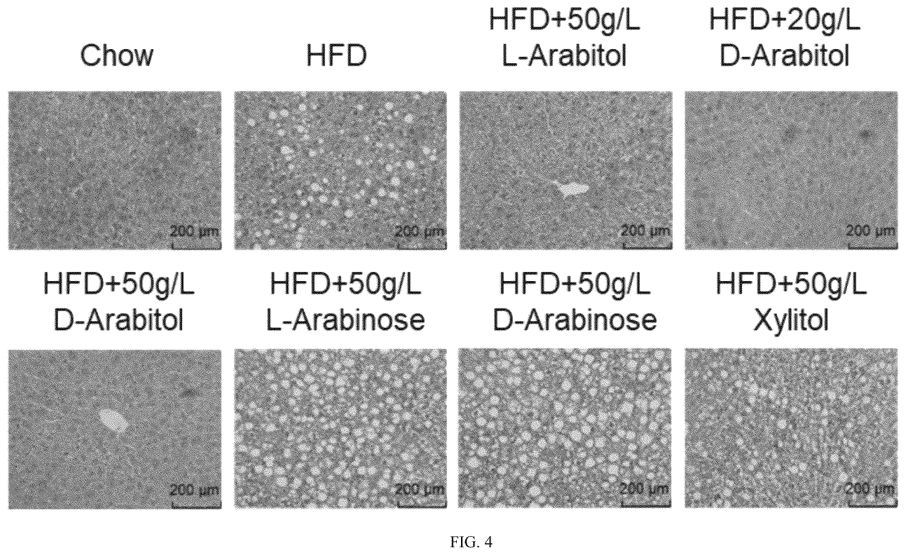

As shown in FIG. 4, compared with mice having the normal diet, in the liver tissue of mice having the high-fat diet, there were a large number of white circular vacuoles, indicating the massive accumulation of lipid droplets. There were no obvious white vacuoles in the liver tissue of mice having the high-fat diet that were administered with 50 g/L or 20 g/L D-arabitol. In mice having the high-fat diet that were administered with 50 g/L L-arabitol, there were no significant white vacuoles, and liver cell structures disappeared, indicating a liver damage. Therefore, the overall improvement effect of L-arabitol was inferior to the overall improvement effect of D-arabitol at a same dose. In conclusion, D-arabitol can alleviate the steatosis of a mouse liver tissue and the liver damage induced by the high-fat diet.

### 5. Aminotransferase

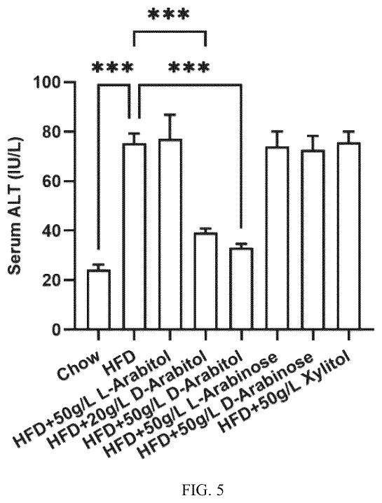

As shown in FIG. 5, an ALT level in serum of mice fed with the high-fat diet for 12 weeks significantly increased (P<0.001). The 20 g/L D-arabitol (P<0.001) and 50 g/L D-arabitol (P<0.001) could significantly reduce a serum ALT concentration in mice having the high-fat diet, and there was no significant change in other groups. Those skilled in the art can know that ALT mainly exists in liver cells and will be released into the blood in large quantities when the liver is damaged, and an ALT level in the blood is a sensitive marker of liver cell damage and is an important indicator to evaluate the liver function impairment. It can be seen that D-arabitol has an excellent protective effect for a liver function in mice with a high-fat diet in a specified dose-dependent manner.

Based on the above experiments, it can be determined that D-arabitol has an excellent lipid-lowering and hepatoprotective effect, and has a prospect of being developed into a drug or health care product or food for lipid-lowering and hepatoprotection.

### Example 2

A lipid-lowering method was provided, including: a drug was orally administered to or injected into a patient with a high blood or liver lipid level, where an active ingredient of the drug was D-arabitol. After the drug with D-arabitol as an active ingredient was orally administered to or injected into the patient with a high blood or liver lipid level, a blood or liver lipid level in the patient decreased effectively.

### Example 3

A hepatoprotection method was provided, including: a drug was orally administered to or injected into a patient with a liver damage caused by a high blood or liver lipid level, where an active ingredient of the drug was D-arabitol. After the drug with D-arabitol as an active ingredient was orally administered to or injected into the patient with a liver damage caused by a high blood or liver lipid level, a liver function in the patient was improved effectively.

### Example 4

A lipid-lowering method was provided, including: an individual with a high blood or liver lipid level was allowed to take a health care product or a food, where the health care product or the food included D-arabitol as an active ingredient. After the individual with a high blood or liver lipid level took the health care product or the food with D-arabitol as an active ingredient, a blood or liver lipid level in the individual decreased effectively.

### Example 5

A hepatoprotection method was provided, including: an individual with a liver damage caused by a high blood or liver lipid level was allowed to take a health care product or a food, where the health care product or the food included D-arabitol as an active ingredient. After the individual with a liver damage caused by a high blood or liver lipid level took the health care product or the food with D-arabitol as an active ingredient, a liver function in the individual was improved effectively.

The above examples are provided to specifically introduce the substantive content of the present disclosure, but those skilled in the art should understand that the protection scope of the present disclosure should not be limited to these specific examples.

What is claimed is:

1. A lipid-lowering method, comprising: orally administering or injecting a drug to or into a patient with a high blood or liver lipid level, wherein an active ingredient of the drug is D-arabitol; or comprising: allowing an individual with the high blood or liver lipid level to take a health care product or a food, wherein the health care product or the food comprises the D-arabitol as an active ingredient.

2. A hepatoprotection method, comprising: orally administering or injecting a drug to or into a patient with a liver damage caused by a high blood or liver lipid level, wherein an active ingredient of the drug is D-arabitol; or comprising: allowing an individual with the liver damage caused by the high blood or liver lipid level to take a health care product or a food, wherein the health care product or the food comprises the D-arabitol as an active ingredient.

3. A drug or health care product or food for lipid-lowering and hepatoprotection, comprising D-arabitol as an active ingredient and an adjuvant acceptable in the drug or health care product or food.
